(19) United States
(12) Patent Application Publication
(10) Pub. No.: US 2026/0293826 A1
(43) Pub. Date: Oct. 1, 2026

(54) AI-Optimized Intelligent Pollination System and Method Based on Sound Wave

(71) Applicant: Xinxin Shan, Surrey (CA)

(72) Inventor: Xinxin Shan, Surrey (CA)

(73) Assignee: LED Smart Inc, Surrey, BC (CA)

(21) Appl. No.: 19/093,998

(22) Filed: Mar. 28, 2025

(51) Int. Cl. A01H 1/02; G06F 3/16

(52) U.S. Cl. CPC A01H 1/027; G06F 3/165

(57) ABSTRACT

The present invention relates to an AI-optimized intelligent pollination system and method utilizing sound wave technology to enhance pollen dispersal and fertilization. The system comprises a sound wave generation module, AI-driven environmental sensors, a control unit with machine learning capabilities, and a deployment unit. The method involves collecting environmental data, analyzing it through AI algorithms, and dynamically adjusting sound wave frequencies to improve pollination efficiency. This invention provides a scalable and adaptable solution for increasing pollination success in agriculture and controlled environments.

### CROSS-REFERENCE TO RELATED APPLICATIONS

US PATENT DOCUMENTS
US20170042102A1 February 2017 Safreno
US20160353661A1 December 2016 Caldeira et al.
US20180065749A1 March 2018 Cantrell et al.
US20230157231A1 May 2023 ELGRABLI et al.

### OTHER PUBLICATION

WO1991011904A1 August 1991 RÖNNQVIST et al.
WO2017164544A1 September 2017 KANG et al.

### BACKGROUND OF THE INVENTION

Pollination plays a crucial role in plant reproduction and food production. Traditional pollination methods rely on natural pollinators such as bees or manual human intervention. However, environmental challenges, climate change, and the decline of pollinator populations have led to inefficiencies in natural pollination. Existing artificial pollination methods are labor-intensive, inconsistent, and lack real-time optimization.

There is a need for a system that can enhance pollination efficiency using advanced technologies, such as AI-based sound wave modulation, to stimulate pollen dispersal and increase fertilization rates.

### SUMMARY OF THE INVENTION

The present invention relates to an AI-optimized intelligent pollination system and method utilizing sound waves to enhance pollen dispersal and fertilization efficiency. The system integrates artificial intelligence algorithms to analyze environmental conditions in real-time and dynamically adjust sound wave frequencies, amplitudes, and patterns to facilitate effective pollen release and transfer. The system includes a sound wave generation module that emits optimized frequencies tailored to the characteristics of different plant species. AI-driven environmental sensors continuously collect data on parameters such as humidity, temperature, wind conditions, and pollen availability. The control unit, incorporating AI optimization algorithms, processes this data and modulates the sound wave parameters accordingly to maximize pollination effectiveness. The system is designed for deployment in various agricultural environments, including greenhouses, controlled indoor farms, and open fields, ensuring widespread adaptability and efficiency in enhancing pollination rates.

The invention also incorporates an ultrasonic misting method (ultrasound atomization) to further enhance pollination efficiency, particularly in controlled indoor environments. In this method, an ultrasonic transducer generates high-frequency vibrations, converting a pollen-mixed solution into a fine mist or aerosol. This mist is gently dispersed into the flowering zone within environments such as greenhouses or vertical farms. The tiny aerosolized droplets carrying pollen uniformly deposit onto flower stigmas, effectively facilitating pollination. This technique complements the system's sound-wave-based pollen dispersal by providing targeted and uniform pollen delivery directly to plant reproductive structures.

Through real-time adaptability, the AI-based control mechanism ensures optimal pollination under diverse environmental conditions by continuously refining its operational parameters. The system reduces dependency on natural pollinators and manual pollination methods, making it a scalable and efficient solution for modern agricultural applications.

### BRIEF DESCRIPTION OF DRAWINGS

FIG. 1: A system block diagram of the AI-optimized intelligent pollination system.

FIG. 2: A flowchart illustrating the AI-based pollination process.

FIG. 3: An exemplary configuration of the sound wave emission and control unit.

FIG. 4: A schematic representation of pollen movement influenced by sound waves.

### DETAILED DESCRIPTION OF THE INVENTION

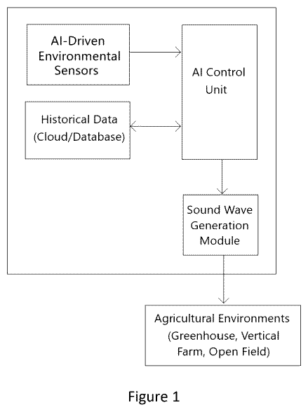

Referring to FIG. 1, the AI-optimized intelligent pollination system comprises multiple interconnected modules designed to enhance pollination efficiency through the use of sound wave technology. The system includes a sound wave generation module configured to produce waves within a predefined frequency range to induce pollen release and dispersal, with the frequency and amplitude adjusted according to plant species, pollen structure, and prevailing environmental conditions. AI-driven environmental sensors collect data relating to temperature, humidity, airflow, and light levels, which is then transmitted to an AI control unit for processing. This control unit employs algorithms to dynamically optimize sound wave parameters and refines these strategies over time by learning from historical pollination patterns. A deployment unit facilitates the placement of the system in various agricultural environments, including greenhouses, vertical farms, and open fields, with a modular design that accommodates different pollination requirements.

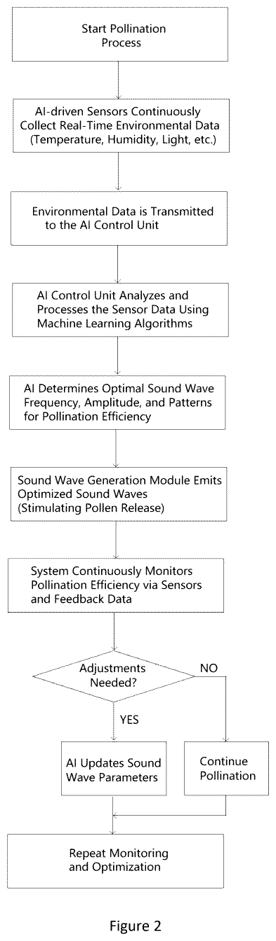

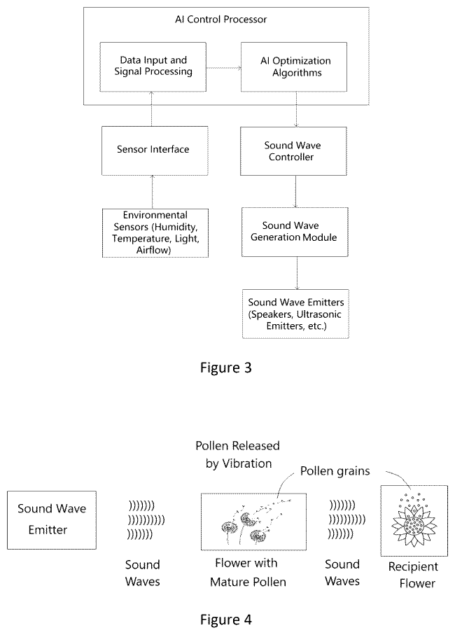

Referring to FIG. 2, the operational process is depicted in a flowchart illustrating how environmental sensors gather real-time data on pollination conditions. The AI control unit analyzes this data to determine optimal sound wave frequencies and intensities for promoting effective pollen dispersal. The sound wave generation module emits the optimized waves, and the system continually monitors pollination efficiency, making further adjustments as needed. FIG. 3 presents an exemplary configuration of the sound wave emission and control unit, showing the interconnections between the sensors, the control unit, and the generation module. Referring finally to FIG. 4, a schematic representation of pollen movement under the influence of sound waves illustrates how targeted wave frequencies enhance pollen release and facilitate its distribution within the growing environment.

1. An AI-optimized intelligent pollination system comprising:
At least one sound wave generation module configured to produce optimized frequencies for pollen release and dispersal;
A plurality of environmental sensors configured to monitor pollination conditions;
An AI control unit configured to process sensor data and dynamically adjust sound wave parameters; and
A deployment unit configured to distribute and install the system in agricultural environments.

2. The system of claim 1, wherein the sound wave generation module operates within a frequency range of 50 Hz to 5 kHz for optimal pollen dispersal.

3. The system of claim 1, wherein the AI control unit employs machine learning algorithms to refine pollination strategies over time based on historical and real-time environmental data.

4. The system of claim 1, wherein the environmental sensors include at least one of humidity, temperature, wind speed, air pressure, light intensity, and pollen concentration sensors.

5. The system of claim 1, wherein the AI control unit is further configured to predict pollination success rates based on environmental data and adjust sound wave emissions accordingly.

6. The system of claim 1, wherein the deployment unit comprises a network of distributed pollination devices, each capable of communicating with a central AI server for synchronized operation.

7. The system of claim 1, wherein the AI control unit receives weather forecast data to preemptively adjust pollination parameters based on anticipated environmental changes.

8. The system of claim 1, wherein the sound wave generation module varies the directionality of emitted sound waves to optimize pollen distribution in multi-layered plant environments.

9. The system of claim 1, wherein the AI control unit is integrated with a wireless communication network, enabling remote monitoring and control of the pollination system.

10. The system of claim 1, wherein the AI control unit adjusts the sound wave intensity based on real-time pollen dispersion feedback detected by optical or air-sampling sensors.

11. The system of claim 1, wherein the sound wave generation module is housed in a weatherproof and vibration-resistant enclosure for stable operation in outdoor environments.

12. The system of claim 1, wherein the AI control unit is further configured to coordinate with natural pollinators by detecting the presence of bees, butterflies, or other insects and adjusting pollination strategies accordingly.

13. A method for AI-optimized intelligent pollination, comprising:
Collecting real-time environmental data using AI-driven sensors;
Analyzing the data to determine optimal sound wave parameters for pollen dispersion;
Emitting sound waves at predefined frequencies and amplitudes to enhance pollination; and
Continuously adjusting the sound wave parameters based on feedback from environmental sensors.

14. The method of claim 2, wherein the sound waves are modulated based on the specific plant species and pollen structure to maximize pollination efficiency.

15. The method of claim 2, wherein the system operates in an automated mode or an operator-controlled mode, allowing for manual adjustments when necessary.

16. The method of claim 2, wherein the sound waves are emitted in pulsed or continuous waveforms depending on pollen adhesion characteristics.

17. The method of claim 2, wherein the sound waves are emitted in a frequency-modulated pattern to enhance resonance effects and increase pollen movement efficiency.

18. The method of claim 2, wherein the system provides real-time alerts and data visualization dashboards to monitor pollination progress remotely.

19. A non-transitory computer-readable medium storing instructions that, when executed by a processor, cause an AI control unit to:
Receive environmental sensor data related to pollination conditions;
Analyze the sensor data using machine learning algorithms to determine optimal sound wave parameters;
Generate control signals to modulate sound wave frequencies and amplitudes for improved pollination efficiency; and
Continuously refine pollination strategies based on historical data and real-time feedback.

20. The non-transitory computer-readable medium of claim 3, wherein the AI model is trained on historical pollination data collected from various environmental conditions to improve prediction accuracy.
